(19) United States
(12) Patent Application Publication
(10) Pub. No.: US 2024/0398506 A1
(43) Pub. Date: Dec. 5, 2024

(54) A SYSTEM AND METHOD FOR ASSIGNING A MEDICAL ASSET TO A ROOM USING REAL-TIME WI-FI LOCALIZATION

(71) Applicant: KONINKLIJKE PHILIPS N.V., EINDHOVEN (NL)

(72) Inventors: ALEXANDER SEBASTIAN FURNICA, EINDHOVEN (NL); SUPRIYO CHATTERJEA, EINDHOVEN (NL)

(21) Appl. No.: 18/691,065

(22) PCT Filed: Sep. 7, 2022
(86) PCT No.: PCT/EP2022/074780

§ 371 Date: Mar. 12, 2024

Related U.S. Application Data
(60) Provisional application No. 63/243,237, filed on Sep. 13, 2021.

(30) Foreign Application Priority Data
Apr. 29, 2022 (EP) 22170674.0

(51) Int. Cl. A61B 90/98; G16H 40/20

(52) U.S. Cl. CPC A61B 90/98; G16H 40/20

(57) ABSTRACT

A method and system for assigning an asset to a room in a medical environment using a Wi-Fi based real-time locating system. A room is fingerprinted thereby to determine a first representative location of the room and a threshold distance. The fingerprinting comprises determining a first representative location of the room using location data for a first time period (e.g. 24 hours) and a set of second representative locations of the room using the location data for second time periods (e.g. 1 hour). The threshold distance is found by comparing the first representative location to the set of second representative locations. A current representative location is found for an asset using location data of the asset and is compared to a range of locations attributable to the fingerprinted room. The range of locations attributable to the room is based on the threshold distance. If the current representative location is within the range of locations attributable to the room, the asset is assigned to the room.

### FIELD OF THE INVENTION

The present invention relates to a real-time Wi-Fi location system in a medical/clinical environment and a method of asset tracking by means of real-time Wi-Fi location systems.

### BACKGROUND OF THE INVENTION

As populations around the world age, and the number of admitted patients increases, hospitals are increasingly under pressure to make better use of their resources which include staff, equipment and all medical facilities. Effective planning of resources is crucial for improving hospital productivity, quality and containing or reducing costs. One way in which hospitals can look to reduce costs and improve efficiency is through optimization of physical asset inventory and use. This applies especially for moveable assets such as infusion pumps and wheelchairs.

In order to achieve asset optimization, real-time location systems (RTLS) can be used. More specifically, using RTLS can allow for effective search of assets and allow collection of data on usage, movement and behavior. This allows for data-enabled decision making when it comes to optimizing inventory of assets. For example, calculating utilization for a group of assets and noticing that utilization is very low could mean that there is too much inventory.

Real-Time Location Systems (RTLS) provide immediate or real-time tracking and management of medical equipment, staff and patients. This type of solution enables healthcare facilities to capture workflow efficiencies, reduce costs, and increase clinical quality. RTLS solutions comprise various tags and badges, platforms (Wi-Fi, Infrared, Ultrasound, and others), hardware infrastructure (readers & exciters) and other components (servers, middleware & end-user software).

Typically, an RTLS solution consists of specialized fixed location sensors receiving wireless signals from small ID badges or tags attached to equipment or persons. Each tag transmits its own unique ID in real time, and depending on the technology chosen, the system locates the tags and therefore the location of the tagged entities. Depending on the solution, varying degrees of granularity can be achieved. Basic RTLS solutions can enable tracking in a hospital's unit or floor, whereas clinical-grade systems are able to achieve room, bed, bay, and even shelf-level tracking.

However, using RTLS for asset optimization is easier said than done, especially when the hospital is making use of a less-accurate. Wi-Fi based RTLS. Due to reduced room-level accuracy in Wi-Fi based RTLS, it is hard to determine with confidence whether assets are in storage, and therefore not in use.

### SUMMARY OF THE INVENTION

The invention is defined by the claims.

According to examples in accordance with an aspect of the invention, there is provided a method for assigning an asset to a room in a medical environment using a Wi-Fi based real-time locating system for determining the locations of tags, the method comprising:
fingerprinting a room, wherein the fingerprinting comprises:
receiving a plurality of fingerprinting locations for a first time period from a plurality of tags placed around the room;
calculating a first representative location from the fingerprinting locations for the first time period;
calculating a set of second representative locations from the fingerprinting locations for a plurality of second, shorter, time periods; and
determining a threshold distance for the room by comparing the first representative locations to the set of second representative locations;
receiving a plurality of locations of a tag placed on the asset;
calculating a current representative location from the plurality of locations;
comparing the current representative location to a range of locations attributable to the room, wherein the range of locations is determined using the threshold distance; and
in response to the current representative location being within the range of locations attributable to the room, assigning the asset to the room.

Wi-Fi based real-time locating systems (RTLS) typically have an accuracy of around 2-4 m. This means that an asset may be in a different room than the room indicated by the location from the RTLS. This disadvantage of Wi-Fi based RTLS means that determining whether an asset is in storage or not becomes difficult as the error of the location may indicate the asset is in a nearby room to the storage room, implying the asset is in use even if it is in storage.

Thus, the determination of the threshold distance during fingerprinting provides an indication of the acceptable error of the RTLS. In other words, the threshold distance indicates how far outside a room the Wi-Fi RTLS may show an asset to be whilst still assigning the asset to that room. This reduces erroneous assignments of utilization (i.e. indicating an asset is in use when it is, in fact, in storage).

“Assigning the asset to the room” means making an assessment that the asset is in the room. i.e. assigning data to the asset which indicates that the asset is in the room. This data can then be used to determine a level of utilization of the asset if the asset is known to be not used when located in the room.

The representative location of a plurality of locations may be calculated by determining a centroid, a mean, a median, a weighted average (e.g. latest locations weighted higher), a geometric mean or other averaging tools on the locations received from the RTLS system.

The first and/or second representative locations may in a preferred implementation be centroids of the corresponding fingerprinting locations.

Similarly, the current representative location may be a centroid of the corresponding plurality of locations.

Comparing the current representative location to a range of locations attributable to the room may comprise calculating the distance between the current representative location and the first representative location, wherein the asset is assigned to the room if the calculated distance is smaller than the threshold distance.

The room may be a storage room for storing the asset.

The method may further comprise calculating a utilization of the asset over a time frame by determining the percentage of time the asset has spent in the storage room over the time frame.

A current representative location may be calculated every third time period, wherein the third time period is a time resolution of the Wi-Fi based real-time locating system.

This provides a sliding window for the calculations of the current representative location, thereby improving the granularity of the central centroid measurements.

A combination of the features above thus provides a method for tracking an asset in a medical environment using a Wi-Fi based real-time locating system for determining the locations of tags, the method comprising:
fingerprinting a storage room, wherein the fingerprinting comprises:
receiving a plurality of locations for a first time period from a plurality of tags placed around the storage room;
calculating a first centroid of the locations for the first time period;
calculating a set of second centroids of the locations for a plurality of second, shorter, time periods; and
determining a threshold distance for the storage room by comparing the first centroid to the set of second centroids;
receiving a plurality of locations of a tag placed on the asset;
calculating a third centroid of the locations for a third time period;
calculating the distance between the third centroid and the first centroid; and
in response to the calculated distance being smaller than the threshold distance, assigning the asset to the storage room.

The invention also provides a computer program product comprising computer program code which, when executed on a computing device having a processing system, cause the processing system to perform all of the steps of the method defined above.

The invention also provides a system for assigning an asset to a room in a medical environment using a Wi-Fi based real-time locating system for determining the locations of tags, the system comprising a processor configured to:
fingerprint a room, wherein the processor is configured to fingerprint by:
receiving a plurality of fingerprinting locations for a first time period from a plurality of tags placed around the room;
calculating a first representative location from the fingerprinting locations for the first time period;
calculating a set of second representative locations from the fingerprinting locations for a plurality of second, shorter, time periods; and
determining a threshold distance for the room by comparing the first representative locations to the set of second representative locations;
receive a plurality of locations of a tag placed on the asset;
calculate a current representative location from the plurality of locations;
compare the current representative location to a range of locations attributable to the room, wherein the range of locations is determined using the threshold distance; and
in response to the current representative location being within the range of locations attributable to the room, assign the asset to the room.

The first and/or second representative locations may be centroids of the corresponding fingerprinting locations.

The current representative location may be a centroid of the corresponding locations.

The processor may be configured to compare the current representative location to a range of locations attributable to the room by calculating the distance between the current representative location and the first representative location, wherein the asset is assigned to the room if the calculated distance is smaller than the threshold distance.

The room may be a storage room for storing the asset.

The processor may be further configured to calculate a utilization of the asset over a time frame by determining the percentage of time the asset has spent in the storage room over the time frame.

A current representative location may be calculated every third time period, wherein the third time period is a time resolution of the Wi-Fi based real-time locating system.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 shows a known CenTrak RTLS system;

FIG. 2 shows the steps for fingerprinting; and

FIG. 3 shows a method for assigning assets to a room.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides a method and system for assigning an asset to a room in a medical environment using a Wi-Fi based real-time locating system. A room is fingerprinted thereby to determine a first representative location of the room and a threshold distance. The fingerprinting comprises determining a first representative location of the room using location data for a first time period (e.g. 24 hours) and a set of second representative locations of the room using the location data for second time periods (e.g. 1 hour). The threshold distance is found by comparing the first representative location to the set of second representative locations. A current representative location is found for an asset using location data of the asset and is compared to a range of locations attributable to the fingerprinted room. The range of locations attributable to the room is based on the threshold distance. If the current representative location is within the range of locations attributable to the room, the asset is assigned to the room. i.e. assessed as being in the room.

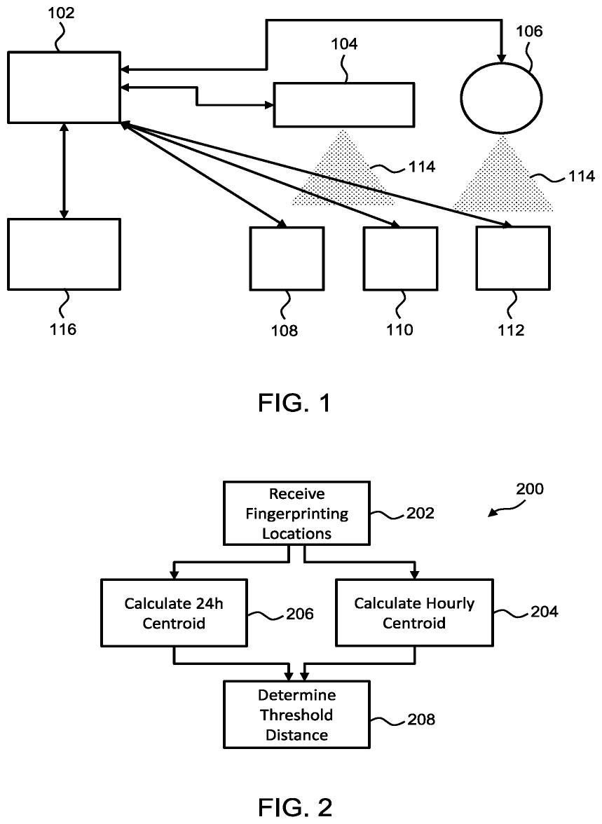

One example of a known RTLS solution is the so-called CenTrak (Trademark) RTLS. FIG. 1 shows a CenTrak RTLS system. The CenTrak RTLS has been used in several studies to automatically record and analyze workflow characteristics of emergency departments and other hospital departments, and this approach is also used by the asset tracking system of the applicant named “PerformanceFlow” in commercial pilots.

The CenTrak system (www.centrak.com) is a clinical-grade, commercially available, system that is fully CE and FCC certified and has been applied in over 750 hospitals world-wide. The CenTrak RTLS solution comprises beacons (comprising “Monitors” 106 and “VirtualWalls” 104) that emit an infrared (IR) code 114 representing a room or corridor, and patient tags 108, asset tags 110 and staff badges 112 that can detect the IR code 114 and transmit this code together with their own ID via a router (“Star”) 102 to a server 116. The beacons 104 and 106 are battery-operated, and hence do not need to be connected to the mains, and can be positioned wherever location data is needed. Since the IR signals 114 do not pass through walls, the room location is detected with certainty. The Stars 102. Monitors 106 and Virtual Walls 104 are ceiling-mounted, the staff tags 112 and the patient tags 108 are worn using regular hospital wristbands and the asset tags 110 are attached to the assets/devices to be tracked. All assets/devices are designed specifically for hospital use.

The tags 108, 110 and 112 communicate with the Star 102 via an RF link using a 900 MHz channel. Similarly, the Virtual Walls 104 and the Monitors 106 communicate with the Star 102 also via an RF link using a 900 MHz channel. Typically, the Star 102 communicates with the Server 116 via a wired Ethernet connection.

The Monitors 106. Virtual Walls 104 and Stars 102 can be placed in emergency rooms, hallways, exam rooms, operation rooms or other areas of the hospital according to the context and purpose of the RTLS implementation.

Wi-Fi based RTLS operates similarly to the IR system used by CenTrak RTLS. The main difference is that the individual tags communicate with Wi-Fi access points (APs) instead of monitors. These APs triangulate the position of the tag based on the signal strength and report the location to the Centrak system to create one single stream of location information.

Wi-Fi based localization is inaccurate compared to infrared. The system computes (X, Y) coordinate pairs for each tag using the relative signal strength indicator (RSSI) that a tag reports for every nearby access point. However, this approach alone is insufficient because it does not take into account specific environmental conditions, such as walls, layout of the rooms, number of objects within a room or movements of other objects or people in the vicinity.

This makes room-based utilization metrics difficult to trust, when using Wi-Fi based RTLS, due to the likelihood of rooms being incorrectly reported.

It is an aim of the current invention to solve the aforementioned issues in Wi-Fi based RTLS and make it possible to create more trustworthy room-based metrics, like utilization, for Wi-Fi-based RTLS systems.

Most often, assets have specific storage rooms dedicated throughout the hospital. In these areas, it can be assumed that the assets are not in use and are therefore a good proxy for calculating utilization. More specifically, the calculation is:

$$u = 1 - s$$

Where u is the utilization percentage of an asset for a given time frame and s is the percentage of time the asset has spent in a storage room for the same time frame.

In order to calculate s, a fingerprinting approach can be used where RTLS tags are stored in different areas of the room for a set period of time. This period of time can range from 24 hours to multiple weeks depending on how dynamic the observed signals are. The following examples will be using a 24 hour data collection period.

Note that in this case, the term “fingerprinting” is used in the networking and cybersecurity sense of the word. In other words, fingerprinting refers to a set of information that can be used to identify network protocols, operating systems, hardware devices, software among other things. The word should be distinguished from fingerprinting as referred in technology to distinguish people from each other using their biological fingerprint. The fingerprinting step may be considered to be a calibration phase for the system

FIG. 2 shows the steps for fingerprinting 200. In a time period of 24 hours, the RTLS tags will report many different (X. Y) coordinate pairs indicating fingerprinting locations. The Wi-Fi based RTLS will receive the coordinate pairs in step 202. The coordinate pairs are grouped into hourly datasets and a centroid is calculated per storage room per hour in step 204. A centroid is then calculated across the entire 24 hours in step 206 and the distance of the hourly centroids are compared to the centroid of the full 24 hours. This is done to understand what an acceptable distance threshold would be for a tag's hourly centroid to be assigned to the storage. As such, a threshold distance is determined in step 208 based on the comparison. The threshold distance is for example just large enough that all of the hourly centroids fall within the area defined by the threshold distance from the 24 hour centroid. A margin may be added to the threshold distance.

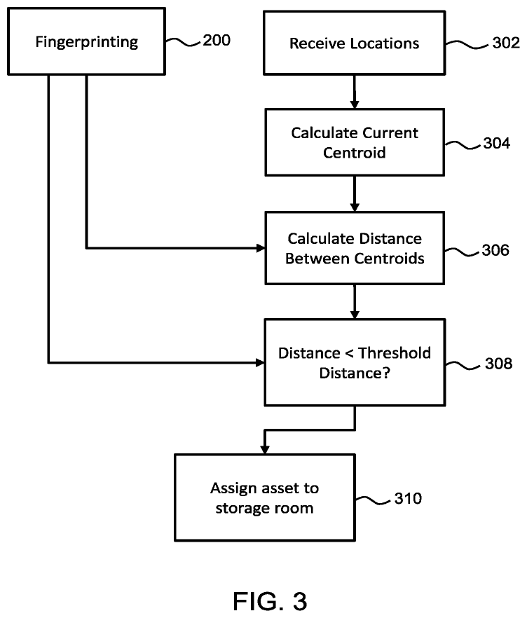

FIG. 3 shows a method for assigning assets to a room. In use, locations are received for each tag in step 302 and hourly centroids are calculated for each tag in step 304. The hourly centroids are compared to the centroids of multiple storage rooms by calculating their distances in step 306. Each storage room is represented by its long term (24 hour in this case) centroid position as determined during the fingerprinting step.

In step 308, should a distance be small enough (i.e. below the threshold distance calculated in the fingerprinting step 200) for a particular storage room, then the asset is assigned to that storage room for the hour in step 310.

A variation on the hourly centroid approach uses a sliding window to allow for finer granularity dependent upon the resolution of the Wi-Fi reports. The sliding window approach is a similar concept to determining the moving average of the location data. A set of location data is used to determine the centroid at a first time and, when a new location coordinate pair is available, a new set of location data is used to determine a new centroid, where the new set of location data is the previous set of location data “shifted forward” in time to include the new location coordinate pair whilst keeping the same size as the previous set.

The resolution of the Wi-Fi reports is a resolution with respect to time. It is typically a user-adjustable feature which indicates how often the Wi-Fi RTLS provides updates on the location. However, the resolution directly impacts battery life. It has been found that time resolutions much shorter than five minutes lead to a reduction in battery-life that make Wi-Fi RTLS infeasible, even if they provide a finer granularity.

The challenge in using hourly centroids to represent asset locations is that any movement out of a storage room will only be detected at the next hourly centroid report. This will create a distorted result that does not reflect reality. In particular, the asset is still not tracked as being in use, pending the next updated position report, despite this asset movement.

The sliding window approach mentioned above resolves this limitation. A centroid is created every T minutes, where T is the time resolution of the Wi-Fi system (and is shorted than the hour period). As soon as an asset is moved out of a storage room, the new position will be reported within T minutes, and this will result in the average centroid position for the preceding hour moving out of the range of positions attributed to the storage room. Thus, within T minutes, the movement out of the storage room can be detected even though the position is a 1 hour average.

In some cases, it is possible that the change in room is not immediately identifiable with the next report. However, the sliding window approach provides a higher likelihood that the move is identified earlier than if centroids were only calculated every hour. Additionally, the delay in identifying the removal of an asset from a storage room may be offset by the delay in identifying the return of the asset to the storage room when calculating the utilization of the asset.

For example, in the case where the Wi-Fi system reports every 5 minutes and the overall desired time period (the 1 hour in this case) involves a number a of centroids being created, that would make the centroid calculation:

$$C {x}_{i , t} = \frac{1}{a} {\sum }_{i - a}^{i} x$$

$$C {y}_{i , t} = \frac{1}{a} {\sum }_{i - a}^{i} y$$

Where i is the count indicative of a coordinate pair at time t. Cx_{i,t} is the centroid's X coordinate at time t. Cy_{i,t} is the centroid's Y coordinate at time t and a is the amount of steps in the past that you want to look at. In this example, a would be equal to 12 (i.e. 60 minutes per hour divided in 5 minute intervals).

As such, the centroids still comprise an hour of data whilst an updated centroid is provided every 5 minutes. This increases the rate at which the centroids are updated. Even though there may be a delay between an asset leaving the storage room and the asset not being assigned to the storage room due to the sliding window approach, there will also be a similar delay when the asset returns to the storage room. As such, both delays may cancel each other out and the percentage time of the asset in the storage room will not be significantly affected.

The method for assigning assets to a room can be used in a specific department or the entire organization. e.g. the Radiology Department of a hospital, the entire hospital, an office building etc.

The proposed method provides a technique to use the spread of X and Y coordinate reports of Wi-Fi-based RTLS over time to better estimate the time spent by assets in storage rooms.

In the example above, there is a single location obtained from a 24 hour time period (a first time period), hourly locations (a second time period) to enable a threshold distance to be determined, and a 5 minute sliding window (a third time period) for the tag locations. However, these are only examples.

More generally, any suitable first time period may be used for the plurality of fingerprinting locations to obtain a first representative location, and any suitable shorter second time period may be used for obtaining the second representative locations from which the threshold distance is determined. The tag location. i.e. a current representative location may be received at a rate corresponding to any desired third time period, shorter than the second time period.

The first representative location, the second representative locations and/or the current representative location may be calculated by calculating the mean, the median, the centroid, a time weighted average etc. of the corresponding locations.

The range of locations attributable to the room may be determined by determining a region around the first representative location (e.g. a circle with the radius being the threshold distance). Alternatively, the range of locations may be a region including the whole area within the room and an additional region outside the room (e.g. an additional distance equal to the threshold distance, or a percentage thereof, from the edges of the room). In some cases, the range of locations may be a circle around the physical center of the room with radius of the threshold distance (or multiple/fraction thereof).

If the range of locations is a circle of radius r (where r is dependent on the threshold distance) from a particular location (e.g. first representative location), comparing a current representative location to the range of locations may comprise determining the difference between the current representative location and the particular location and comparing the difference to the radius. Other methods could also be used (e.g. using a search table comprising all the locations within the range of locations).

The range of locations may be used for all assets in a room or different regions could be used for different assets. This is because different assets may be placed consistently in different parts of the room.

The room may be a storage room or any other kind of room. For example, the room could be an operating room or an emergency room. Thus, assigning the asset to these rooms may provide information on who uses these assets the most and where these assets are most used. This information may further inform the user where would be best to store certain assets to keep them close to where they are most used.

The first time period could be, for example 4 hours. 10 hours. 24 hours, various days or possibly even a week. Of course, the longer the first time period, the more accurate the first representative location will be.

The second time period could be, for example 30 minutes. 1 hour. 2 hours, 4 hours or possible even a whole day, as long as it is shorter than the first time period. The second time period should be at least half of the first time period such that at least two second representative locations can be determined within the first time period.

The threshold distance may be based on the modulus of the difference between the average of the second representative locations and the first representative location, or a multiple/fraction thereof. The particular method for determining the threshold distance using the first and second representative locations may depend on the particular accuracy needs or personal preference of the user. For example, if two tracked rooms are relatively near each other, they may need relatively smaller threshold distances such that an asset cannot be assigned to both rooms simultaneously. The same reasoning may also be applied to the determination of the range of locations, where the exact range and/or shape of the range may depend on the particular needs of the user and the nearby environment (e.g. tracked rooms nearby).

As a result of the inherent inaccuracy of the location system, an asset may be determined to be in the storage room when in fact it is being used in an adjacent room. This situation may be detected using a temporal analysis. For example, if the asset only appears to be in the storage room area for a short time period between longer periods in an adjacent room, this can be ignored if that short time period is less than a threshold time it takes to move the asset into and out of the storage room. It means the asset only appeared to be in the storage room because of the inaccuracy of the positioning system. Thus, temporal analysis can improve the accuracy of the assignment of the asset to the storage room or not.

The skilled person would be readily capable of developing a processor for carrying out any herein described method. Thus, each step of a flow chart may represent a different action performed by a processor, and may be performed by a respective module of the processor.

As discussed above, the system makes use of processor to perform the data processing. The processor can be implemented in numerous ways, with software and/or hardware, to perform the various functions required. The processor typically employs one or more microprocessors that may be programmed using software (e.g., microcode) to perform the required functions. The processor may be implemented as a combination of dedicated hardware to perform some functions and one or more programmed microprocessors and associated circuitry to perform other functions.

Examples of circuitry that may be employed in various embodiments of the present disclosure include, but are not limited to, conventional microprocessors, application specific integrated circuits (ASICs), and field-programmable gate arrays (FPGAs).

In various implementations, the processor may be associated with one or more storage media such as volatile and non-volatile computer memory such as RAM, PROM, EPROM, and EEPROM. The storage media may be encoded with one or more programs that, when executed on one or more processors and/or controllers, perform the required functions. Various storage media may be fixed within a processor or controller or may be transportable, such that the one or more programs stored thereon can be loaded into a processor.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality.

Functions implemented by a processor may be implemented by a single processor or by multiple separate processing units which may together be considered to constitute a “processor”. Such processing units may in some cases be remote from each other and communicate with each other in a wired or wireless manner.

The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

If the term “adapted to” is used in the claims or description, it is noted the term “adapted to” is intended to be equivalent to the term “configured to”. If the term “arrangement” is used in the claims or description, it is noted the term “arrangement” is intended to be equivalent to the term “system”, and vice versa.

Any reference signs in the claims should not be construed as limiting the scope.

1. A method for assigning an asset to a room in a medical environment using a Wi-Fi based real-time locating system for determining the locations of tags, the method comprising:
fingerprinting a room, wherein the fingerprinting comprises:
receiving a plurality of fingerprinting locations for a first time period from a plurality of tags placed around the room;
calculating a first representative location from the fingerprinting locations for the first time period;
calculating a set of second representative locations from the fingerprinting locations for a plurality of second, shorter, time periods; and
determining a threshold distance for the room by comparing the first representative locations to the set of second representative locations;
receiving a plurality of locations of a tag placed on the asset;
calculating a current representative location from the plurality of locations;
comparing the current representative location to a range of locations attributable to the room, wherein the range of locations is determined using the threshold distance; and
in response to the current representative location being within the range of locations attributable to the room, assigning the asset to the room.

2. The method of claim 1, wherein the first and/or second representative locations are centroids of the corresponding fingerprinting locations.

3. The method of claim 1, wherein the current representative location is a centroid of the corresponding plurality of locations.

4. The method of claim 1, wherein comparing the current representative location to a range of locations attributable to the room comprises calculating the distance between the current representative location and the first representative location, wherein the asset is assigned to the room if the calculated distance is smaller than the threshold distance.

5. The method of claim 1, wherein the room is a storage room for storing the asset.

6. The method of claim 5, further comprising calculating a utilization of the asset over a time frame by determining the percentage of time the asset has spent in the storage room over the time frame.

7. The method of claim 1, wherein a current representative location is calculated every third time period, wherein the third time period is a time resolution of the Wi-Fi based real-time locating system.

8. A computer program product comprising computer program code which, when executed on a computing device having a processing system, cause the processing system to perform all of the steps of the method according to claim 1.

9. A system for assigning an asset to a room in a medical environment using a Wi-Fi based real-time locating system for determining the locations of tags, the system comprising a processor configured to:
fingerprint a room, wherein the processor is configured to fingerprint by:
receiving a plurality of fingerprinting locations for a first time period from a plurality of tags placed around the room;
calculating a first representative location from the fingerprinting locations for the first time period;
calculating a set of second representative locations from the fingerprinting locations for a plurality of second, shorter, time periods; and
determining a threshold distance for the room by comparing the first representative locations to the set of second representative locations;
receive a plurality of locations of a tag placed on the asset;
calculate a current representative location from the plurality of locations;
compare the current representative location to a range of locations attributable to the room, wherein the range of locations is determined using the threshold distance; and
in response to the current representative location being within the range of locations attributable to the room, assign the asset to the room.

10. The system of claim 9, wherein the first and/or second representative locations are centroids of the corresponding fingerprinting locations.

11. The system of claim 9, wherein the current representative location is a centroid of the corresponding locations.

12. The system of claim 9, wherein the processor is configured to compare the current representative location to a range of locations attributable to the room by calculating the distance between the current representative location and the first representative location, wherein the asset is assigned to the room if the calculated distance is smaller than the threshold distance.

13. The system of claim 9, wherein the room is a storage room for storing the asset.

14. The system of claim 13, wherein the processor is further configured to calculate a utilization of the asset over a time frame by determining the percentage of time the asset has spent in the storage room over the time frame.

15. The system of claim 9, wherein a current representative location is calculated every third time period, wherein the third time period is a time resolution of the Wi-Fi based real-time locating system.
